(19) United States
(12) Patent Application Publication
(10) Pub. No.: US 2025/0295361 A1
(43) Pub. Date: Sep. 25, 2025

(54) PATIENT TRAINER MIRROR

(71) Applicant: JANSSEN BIOTECH, INC., HORSHAM, PA (US)

(72) Inventors: JUSTIN MELLINGER, PHILADELPHIA, PA (US); RYAN T. WALSH, DOWNINGTOWN, PA (US); JOHN QUINLAN, SPRING CITY, PA (US)

(21) Appl. No.: 19/107,952

(22) PCT Filed: Aug. 31, 2023
(86) PCT No.: PCT/IB2023/058604

§ 371 Date: Feb. 28, 2025

Related U.S. Application Data
(60) Provisional application No. 63/374,528, filed on Sep. 2, 2022.

(51) Int. Cl. A61B 5/00

(52) U.S. Cl. CPC A61B 5/684; A61B 5/0079; A61B 5/444; A61B 5/445; A61B 5/6898; A61B 5/7267

(57) ABSTRACT

The present invention is a smart mirror device that provides custom guidance to a patient regarding where to place a medical device on their body. The smart mirror device can rely on machine-learning or other software modules to analyze images of the patient and to identify the optimal placement location for the medical device based on those images and other factors such as dermatological conditions of the patient's skin, historical data regarding which locations of the patient's body received an injection from a medical device, medical device limitations, curvature of the patient's body, etc. Upon determining the target location, the smart mirror device can provide guidance to the patient to move the medical device from a detected location to the target location via one or more indicators.

### FIELD

The present invention relates to systems and methods for guiding a patient to locate a medical device on his or her body, and more specifically, to guiding self-placement of medical devices using a machine-learning (ML) model to analyze images of the patient and identify the ideal placement location for the medical device.

### BACKGROUND

Many drugs in development, such as large-molecule biologics, have viscosities, dosage volumes, or delivery profiles that are not amenable to manual injection by needle and syringe. Instead, these drugs may be more effectively administered, and with less disruption to the patient's routine, by relying on on-body devices or auto-injectors such as a wearable injector or an infusion pump.

On-body devices and auto-injectors are used to deliver pharmacological agents into a patient's body in controlled amounts. Beneficially, such medical devices do not rely on the patient to measure out the appropriate dosage. The patient need only apply the medical device to their body and instruct the medical device to begin the dosage regiment.

Although medical devices such as on-body devices and auto-injectors improve ease of use once the medical device has been applied, usability issues exist with respect to applying the medical device on the patient's body in the optimal location and appropriate orientation. Sites such as the abdomen, front thigh, inner thigh, and back of the arm are optimal sites for subcutaneous injection. However, injection site selection is complicated based on a variety of factors such as the variety of patient body shapes and sizes, skin conditions of the patient, pump orientation limitations, the patient's individual treatment history, and therapy protocols of the medical device and/or pharmacological agent being delivered. A further complication may arise with specific medical devices that include a needle that protrudes on the side of the device that touches the patient's skin only when using the device but recedes back into the device otherwise, because the needle cannot easily be viewed by the patient when attempting to locate the device appropriately. Such complications can be overwhelming to patients, and especially so to patients who are technology-phobic or limited in some manner.

If on-body devices and auto-injectors are to be widely accepted and displace manual drug injection techniques, these devices must be easy to apply, use, remove, and dispose. Furthermore, the start-up costs for aspects such as patient training and guidance from a health care provider must be minimized to ensure such devices are economically feasible.

One method of teaching patients how to apply and use on-body devices and auto-injectors includes providing written instructions for use (IFU) or a video recording of a healthcare provider or other knowledgeable individual providing general IFU. General one-size-fits-all IFU, however, can introduce patient errors and hazards with respect to comprehension issues. Moreover, one-size-fits-all IFU fail to address the variety of body shapes and sizes of patients that are relevant to determining where to apply a given device.

Alternatively, a healthcare provider can provide direct custom guidance to each patient to teach them how to appropriately apply and use such devices. Relying on direct interaction between a healthcare provider and patient, however, also presents various issues. For instance, in remote geographical regions, patients may not have convenient access to healthcare facilities and healthcare providers. Moreover, regiments requiring direct guidance from a health care provider may make using on-body medical devices prohibitively expensive. Further, once a patient learns to how to use and apply a given device, they may still require oversight by a health care provider to ensure that they continue to appropriately use the medical device, requiring additional in-person check-ins, which will increase the cost of the treatment regimen.

Accordingly, there exists a need for systems and methods for guiding a patient to apply on-body and auto-injector medical devices in the optimal location and orientation that minimizes health care provider involvement in training and surveillance of the patient, improves the ease of use for patients, reduces the risk of patient error caused by misunderstanding relevant IFU, and provides custom guidance to each patient that considers their unique body shape and size, dermatological conditions, and treatment history.

### SUMMARY

Provided herein is a smart mirror device that provides custom guidance to a patient regarding where to place a medical device on their body that meets the above need. The smart mirror device can be computing device such as a mobile phone or laptop that displays an image and/or video feed of the patient or includes a mirror that enables the patient to view his or her reflection. The smart mirror device can guide self-placement of a medical device using a machine-learning model to analyze images of a patient, identify the optimal placement location for the medical device, and provide guidance to the patient to place the medical device in that location. The smart mirror device can rely on a number of machine-learning or other software modules to identify a target location on the patient's body that takes into account dermatological conditions of the patient's skin, historical data regarding which locations of the patient's body recently received treatment via a medical device, and/or the curvature of the patient's body. Upon identifying this target location, the smart mirror device can generate guidance based on detecting the location of the medical device in one or more images of the patient to direct the patient to move the medical device to the target location. This guidance can be provided to the patient by an indicator of the smart mirror device, such as by objects displayed on a display screen, auditory guidance emitted from speakers, illuminated lights, etc. and/or via indicators of the medical device. Accordingly, the smart mirror device can guide a patient to apply a medical device in an optimal location, providing custom guidance considering the patient's body shape and size, dermatological conditions, and treatment history. By providing direct custom guidance from the smart mirror device, the smart mirror device can improve the ease of use for the patient when using a medical device and minimize risk in misunderstanding how or where to place/orient the medical device without necessitating direct guidance from a health care provider.

In one or more examples, a method for guiding a patient to place a medical device on the patient's body using a computing device comprises: acquiring one or more images of the patient via one or more cameras of the computing device, detecting one or more characteristics of the patient's body in the one or more images, determining a target location for placing the medical device on the patient's body based on the detected one or more characteristics, detecting a location of the medical device, generating guidance for moving the medical device from the detected location of the medical device to the determined target location, and providing the guidance to the patient via one or more indicators of the computing device.

Optionally, the computing device comprises a mirror.

Optionally, the computing device is one of a mobile phone, a laptop, and a tablet.

Optionally, the one or more indicators comprise a display.

Optionally, the method comprises displaying one or more images and/or a video feed of the patient via the display.

Optionally, providing guidance to the patient comprises displaying one or more user interface objects on the display.

Optionally, determining the target location comprises identifying one or more unsuitable areas of the patient's body based on the detected one or more characteristics.

Optionally, the one or more unsuitable areas of the patient's body comprise areas with one or more of inflammation, infection, eczema, cancer, and psoriasis.

Optionally, identifying the one or more unsuitable areas is based in part on how much time has elapsed since the patient last administered an injection from any medical device.

Optionally, identifying the one or more unsuitable areas is based in part on orientation limitations of the medical device.

Optionally, determining the target location comprises generating a photogrammetric model of the patient and identifying the one or more unsuitable areas is based in part on the photogrammetric model.

Optionally, identifying the one or more unsuitable areas comprises determining three dimensional normal surface vectors of the photogrammetric model, and the one or more unsuitable areas comprise areas wherein the three dimensional normal surface vectors are incompatible with one or more orientation limitations of the medical device.

Optionally, identifying the one or more unsuitable areas comprises determining three dimensional gradient surface vectors of the photogrammetric model, and the one or more unsuitable areas comprise areas wherein the three dimensional gradient surface vectors are incompatible with one or more orientation limitations of the medical device.

Optionally, generating the photogrammetric model comprises obtaining a plurality of body reference keypoints in near real-time based on the one or more images.

Optionally, determining the target location comprises identifying a target region on the patient's body based on the body reference keypoints and identifying the target location within the target region based on the one or more unsuitable areas.

Optionally, determining the target location comprises: mapping the target region to the photogrammetric model of the patient, un-mapping the one or more unsuitable areas from the mapped photogrammetric model, and identifying the target location within the mapped photogrammetric model.

Optionally, each of the body reference keypoints corresponds to a body part of the patient. Optionally, one of the body reference keypoints corresponds to the patient's navel.

Optionally, obtaining the plurality of estimated body reference keypoints comprises processing the one or more images using a machine-learning model for estimating pose. Optionally, the machine-learning model is a convolutional neural network model.

Optionally, detecting the location of the medical device comprises extracting a custom set of anthropometric ratios for the patient based in part on the body reference keypoints and inferring the location of the medical device based on the custom set of anthropometric ratios for the patient.

Optionally, generating guidance comprises generating a 2D positioning vector based on the body reference keypoints and the custom set of anthropometric ratios for the patient, and wherein the one or more user interface objects comprise a user interface object corresponding to the 2D positioning vector.

Optionally, detecting the location of the medical device comprises identifying one or more device reference keypoints based on the one or more images.

Optionally, generating guidance comprises generating a 2-dimensional (2D) positioning vector based on the one or more device reference keypoints and the target location, and wherein the one or more user interface objects comprise a user interface object corresponding to the 2D positioning vector on the computing device.

Optionally, generating guidance comprises generating a 3-dimensional (3D) positioning rotation angle based on the one or more device reference keypoints and the target location, and wherein the one or more user interface objects comprise a user interface object corresponding to the 3D positioning rotation angle on the computing device.

Optionally, the 2D positioning vector and/or the 3D positioning rotation angle are generated based in part on data from an accelerometer of the medical device.

Optionally, detecting one or more characteristics of the patient comprises processing the one or more images using a machine-learning model for detecting dermatological conditions.

Optionally, the computing device is communicatively connected to a device of a health care provider.

Optionally, the one or more indicators comprise one or more illuminators and providing guidance to the patient comprises illuminating the one or more illuminators.

Optionally, the one or more indicators comprise one or more speakers, and providing guidance to the patient comprises emitting auditory signals from the one or more speakers.

Optionally, the medical device is a wearable drug delivery system.

Optionally, the wearable drug delivery system comprises a reservoir holding a therapeutic agent and the wearable drug delivery system must be within a specific orientation on the patient to successfully deliver the therapeutic agent.

Optionally, the medical device is an auto-injector.

In one or more examples, an interactive patient guidance system comprises: a medical device, a computing device comprising: one or more cameras, one or more indicators, and one or more processors configured to run instructions to: acquire one or more images of a patient via the one or more cameras, detect one or more characteristics of the patient's body in the one or more images, determine a target location for placing the medical device on the patient's body based on the detected one or more characteristics, detect a location of the medical device, generate guidance for moving the medical device from the detected location of the medical device to the determined target location, and provide the guidance to the patient via the one or more indicators on the computing device.

Optionally, the computing device comprises a mirror.

Optionally, the computing device is one of a mobile phone, a laptop, and a tablet.

Optionally, the one or more indicators comprise a display.

Optionally, the one or more processors are configured to run instructions to display one or more images and/or a video feed of the patient via the display.

Optionally, providing the guidance to the patient comprises displaying one or more user interface objects on the display.

Optionally, determining the target location comprises identifying one or more unsuitable areas of the patient's body based on the detected one or more characteristics.

Optionally, the one or more unsuitable areas of the patient's body comprise areas with one or more of inflammation, infection, eczema, cancer, and psoriasis.

Optionally, identifying the one or more unsuitable areas is based in part on how much time has elapsed since the patient last administered the medical device.

Optionally, identifying the one or more unsuitable areas is based in part on orientation limitations of the medical device.

Optionally, determining the target location comprises generating a photogrammetric model of the patient and identifying the one or more unsuitable areas is based in part on the photogrammetric model.

Optionally, identifying the one or more unsuitable areas comprises determining three dimensional normal surface vectors of the photogrammetric model, and the one or more unsuitable areas comprise areas wherein the three dimensional normal surface vectors are incompatible with one or more orientation limitations of the medical device.

Optionally, identifying the one or more unsuitable areas comprises determining three dimensional gradient surface vectors of the photogrammetric model, and the one or more unsuitable areas comprise areas wherein the three dimensional gradient surface vectors are incompatible with one or more orientation limitations of the medical device.

Optionally, generating the photogrammetric model comprises obtaining a plurality of body reference keypoints in near real-time based on the one or more images.

Optionally, determining the target location comprises identifying a target region on the patient's body based on the body reference keypoints and identifying the target location within the target region based on the one or more unsuitable areas.

Optionally, determining the target location comprises: mapping the target region to the photogrammetric model of the patient, un-mapping the one or more unsuitable areas from the mapped photogrammetric model, and identifying the target location within the mapped photogrammetric model.

Optionally, each of the body reference keypoints corresponds to a body part of the patient. Optionally, one of the body reference keypoints corresponds to the patient's navel.

Optionally, obtaining the plurality of estimated body reference keypoints comprises processing the one or more images using a machine-learning model for estimating pose. Optionally, the machine-learning model is a convolutional neural network model.

Optionally, detecting the location of the medical device comprises extracting a custom set of anthropometric ratios for the patient based in part on the body reference keypoints and inferring the location of the medical device based on the custom set of anthropometric ratios for the patient.

Optionally, generating guidance comprises generating a 2D positioning vector based on the body reference keypoints and the custom set of anthropometric ratios for the patient, and wherein the one or more user interface objects comprise a user interface object corresponding to the 2D positioning vector.

Optionally, detecting the location of the medical device comprises identifying one or more device reference keypoints based on the one or more images.

Optionally, generating guidance comprises generating a 2-dimensional (2D) positioning vector based on the one or more device reference keypoints and the target location, and wherein the one or more user interface objects comprise a user interface object corresponding to the 2D positioning vector on the computing device.

Optionally, generating guidance comprises generating a 3-dimensional (3D) positioning rotation angle based on the one or more device reference keypoints and the target location, and wherein the one or more user interface objects comprise a user interface object corresponding to the 3D positioning rotation angle on the computing device.

Optionally, the medical device comprises an accelerometer and the 2D positioning vector and/or the 3D positioning rotation angle are generated based in part on data from the accelerometer.

Optionally, detecting one or more characteristics of the patient comprises processing the one or more images using a machine-learning model for detecting dermatological conditions.

Optionally, the computing device is communicatively connected to a device of a health care provider.

Optionally, the one or more indicators comprise one or more illuminators and providing guidance to the patient comprises illuminating the one or more illuminators.

Optionally, the one or more indicators comprise one or more speakers, and providing guidance to the patient comprises emitting auditory signals from the one or more speakers.

Optionally, the medical device is a wearable drug delivery system.

Optionally, the wearable drug delivery system comprises a reservoir holding a therapeutic agent and the wearable drug delivery system must be within a specific orientation on the patient to successfully deliver the therapeutic agent.

Optionally, the medical device is an auto-injector.

In one or more examples, a medical device comprises: one or more indicators, and one or more processors, wherein the medical device is communicatively connected to an external processor and the one or more processors of the medical device are configured to: receive one or more instructions from the external processor for providing guidance to a patient for placing the medical device on the patient's body, and provide the guidance to the patient via the one or more indicators of the medical device.

Optionally, the medical device is an auto-injector.

Optionally, the one or more indicators comprise one or more speakers, and providing guidance to the patient comprises emitting auditory signals from the one or more speakers.

Optionally, the one or more indicators comprise one or more illuminators and providing guidance to the patient comprises illuminating the one or more illuminators.

Optionally, the medical device comprises one or more sensors.

Optionally, the one or more sensors comprise an accelerometer.

Optionally, the one or more sensors comprise a gyroscope.

Optionally, the one or more sensors comprise a magnetometer.

Optionally, the medical device is a wearable drug delivery system.

Optionally, the wearable drug delivery system comprises a reservoir holding a therapeutic agent and the wearable drug delivery system must be within a specific orientation on the patient to successfully deliver the therapeutic agent.

Additional advantages will be readily apparent to those skilled in the art from the following detailed description. The aspects and descriptions herein are to be regarded as illustrative in nature and not restrictive. It will be appreciated that any of the variations, aspects, features, and options described in view of the systems apply equally to the methods and vice versa. It will also be clear that any one or more of the above variations, aspects, features, and options can be combined.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

### BRIEF DESCRIPTION OF THE FIGURES

The invention will now be described, by way of example only, with reference to the accompanying drawings, in which:

FIG. 1 shows a system for providing guidance for placing a medical device to a patient via a smart mirror device system, according to one or more examples;

FIG. 2 shows an exemplary smart mirror device, according to one or more examples;

FIG. 3 shows an exemplary software system for providing guidance for placing a medical device to a patient via a smart mirror device, according to one or more examples;

FIG. 4 shows an exemplary method for providing guidance for placing a medical device to a patient via a smart mirror device, according to one or more examples;

FIG. 5 shows an exemplary depiction of a reference image and supplemental images of a patient that can be obtained by a smart mirror device system, according to one or more examples;

FIG. 6 shows an exemplary method for generating a photogrammetric model of a patient, according to one or more examples;

FIG. 7 shows exemplary extracted body reference keypoints for use in providing guidance to place a medical device, according to one or more examples;

FIG. 8 shows an exemplary method for identifying a target location for placing a medical device, according to one or more examples;

FIG. 9 shows an exemplary method for generating positioning guidance for placing a medical device, according to one or more examples;

FIG. 10 shows an exemplary method for providing positioning guidance for placing a medical device, according to one or more examples;

FIG. 11A shows an exemplary auto-injector medical device, according to one or more examples;

FIG. 11B shows an exemplary auto-injector medical device with the injector needle protruding from the backside of the device, according to one or more examples; and

FIG. 12 shows an exemplary computing device, according to one or more examples.

### DETAILED DESCRIPTION

In the following description of the various examples, reference is made to the accompanying drawings, in which are shown, by way of illustration, specific examples that can be practiced. The description is presented to enable one of ordinary skill in the art to make and use the invention and is provided in the context of a patent application and its requirements. Various modifications to the described examples will be readily apparent to those persons skilled in the art and the generic principles herein may be applied to other examples. Thus, the present invention is not intended to be limited to the examples shown but is to be accorded the widest scope consistent with the principles and features described herein.

Systems and methods are described herein for guiding a patient to place a medical device on the patient's body using a smart mirror device. The system can include a smart mirror device and a medical device. The smart mirror device can be a computing device such as a mobile phone or laptop that displays an image and/or video feed of the patient or includes a mirror that enables the patient to view his or her reflection. The smart mirror device can include one or more cameras, a digital processing unit (DPU), and one or more indicators for providing the guidance to the patient. The DPU can include a controller that controls the one or more cameras and the one or more indicators such that the DPU can execute the method of guiding the patient to place the medical device on their body. The DPU can be communicatively connected to the medical device such that the smart mirror device (via the DPU) can obtain information from the medical device and/or control various indicators of the medical device.

The indicators of the smart mirror device can include one or more display screens, one or more speakers, and/or one or more illuminators. The display screens can display user interface objects, such as directional arrows, text guidance, an icon corresponding to the target location for the medical device, etc. The one or more display screens can display visual guidance such as photos, pre-recorded videos, live videos, etc., showing instructions from an individual such as a health care provider. Optionally, the one or more display screens can display visual guidance from another source, such as from a treatment partner, social media videos, etc. The one or more speakers can emit auditory guidance such as pre-recorded messages or real-time guidance from an individual such as a health care provider. The illuminators can include illuminators shaped in particular configurations, such as shaped as a series of arrows, such that when certain illuminators, for instance a “down” arrow illuminator, are illuminated, the patient can understand what guidance is being conveyed. Optionally, the medical device can include indicators such as illuminators, speakers, and/or a device for vibrating the device to provide guidance to the patient when locating the medical device.

The smart mirror device can rely on software, including one or more machine-learning models, for determining a target location and generating guidance for moving the medical device to the target location. To determine a target location, the smart mirror device can acquire images of the patient via one or more cameras. The smart mirror device can detect characteristics of the patient's skin in those images, such as dermatological conditions like inflammation, scarring, etc. The smart mirror device may rely on a machine-learning model trained to detect such dermatological conditions based on images. The smart mirror device can then determine a target location for placing the medical device based on the detected characteristics.

To determine an appropriate target location, the smart mirror device can identify certain areas of the patient's body as unsuitable. For example, a particular location may result in the medical device not properly delivering the drug, (e.g., a “wet delivery”) which can waste an expensive drug product and introduce issues for the patient based on missing a dose of the drug. Identifying unsuitable locations may be based on a determination that a specific area of the patient's body is not a suitable location for placing the medical device after determining the patient's skin in that area has scarring that may make administering the medical device there difficult.

Identifying unsuitable locations may also be based on historical data relating to when and where the patient administered a medical device to their body. The smart mirror device may determine a particular area is not suitable for placing the medical device because the patient recently administered a medical device to that area, such as within the last week.

Identifying unsuitable locations may be based on the patient's curvature. The smart mirror device may generate a photogrammetric model, which clearly illustrates the curvature of the patient's body, based on images of the patient. The smart mirror device may then use the photogrammetric model to determine whether a certain location is suitable for placing the medical device based on that curvature. For instance, an area of the patient's body with skin folds may not be suitable, if the medical device must be in contact with a continuous area of the patient's body. If the medical device has certain orientation limitation, such as necessitating the medical device be oriented vertically with respect to the gravitational vertical, the smart mirror device may take such orientation limitations into consideration and assess whether a given location is suitable based on the orientation limitations of the medical device as compared to the photogrammetric model of the patient.

In one or more examples, the smart mirror device can obtain a plurality of body reference keypoints corresponding to different areas of the patient's body. Obtaining the body reference keypoints can involve processing images of the patient via a machine learning model trained to identify keypoints, such as a left and right shoulder, elbow, wrist, hip, etc. The smart mirror device can use this information to infer a location of the medical device if the patient is holding the medical device in a certain hand, and/or to generate guidance for moving the medical device to the target location.

After determining the target location, the smart mirror device can detect a location of the medical device. The smart mirror device may detect the location of the medical device by locating one or more device reference keypoints in the images of the patient. The smart mirror device may infer a location of the medical device based on custom anthropometric ratios corresponding to the patient's body and the body reference keypoints of the patient.

After locating the medical device, the smart mirror device can generate guidance for moving the medical device to the target location. For instance, the smart mirror device can determine the medical device needs to be moved downward on the patient's body and rotated by an amount such that the device is both located and oriented properly on the patient's body. Such guidance may be based in part on sensors of the medical device, such as a magnetometer, an accelerometer and/or a gyroscope for obtaining the spatial orientation and spatial acceleration data of the medical device.

To provide guidance, the smart mirror device can rely on the indicators of the smart mirror device and/or the medical device. For example, the smart mirror device may display user interface objects on a display screen, emit auditory guidance via speakers, and/or illuminate illuminators in a manner that provides comprehensible guidance to the patient. The smart mirror device may, additionally or alternatively, control indicators on the medical device to provide guidance. For example, the smart mirror device may cause the medical device to flash illuminators or emit beeping sounds that help the user when attempting to locate the medical device in the target location. Upon locating the medical device appropriately, the smart mirror device can provide guidance that informs the patient to adhere the medical device to their body. Optionally, the smart mirror device can also provide guidance to the patient to administer the medical device, such as how to use the device, how to pose while the device is administering a drug, and how to dispose of the device and/or the drug thereafter.

As used herein, the singular forms “a,” “an,” and “the” used in the following description are intended to include the plural forms as well unless the context clearly indicates otherwise. It is to be understood that the term “and/or” as used herein refers to and encompasses any and all possible combinations of one or more of the associated listed items. It is further to be understood that the terms “includes,” “including,” “comprises,” and/or “comprising,” when used herein, specify the presence of stated features, integers, steps, operations, elements, components, and/or units but do not preclude the presence or addition of one or more other features, integers, steps, operations, elements, components, units, and/or groups thereof.

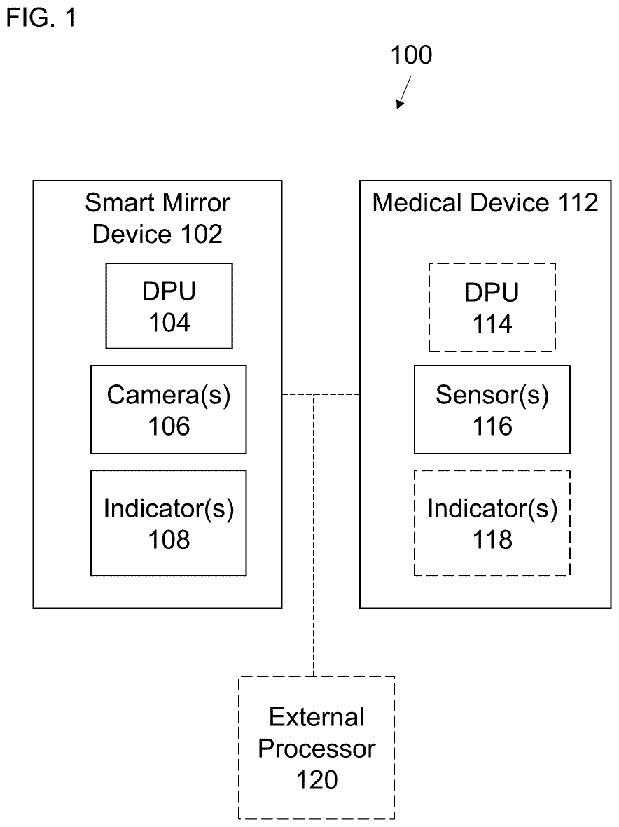

FIG. 1 shows a smart mirror device system 100 for providing guidance for placing a medical device to a patient via a smart mirror device system, according to one or more examples. The smart mirror device system 100 includes a smart mirror device 102 that is communicatively connected to a medical device 112. The smart mirror device 102 can include a digital processing unit (DPU) 104, one or more cameras 106, and one or more indicators 108. The medical device 112 can include one or more sensors 116, and optionally can include a DPU 114 and one or more indicators 118. Optionally, the smart mirror device system 100 includes an external processor 120. According to one or more examples, the smart mirror device system 100 can be used to provide guidance to a patient, such as by an indicator 108 of the smart mirror device 102 or an indicator 118 of the medical device 112, to place the medical device 112 in the optimal location and position on the patient's body.

The camera(s) 106 may capture still and/or video images. The images captured by the camera(s) 106 can be in color or monochrome. Optionally, the camera(s) 106 are a 5MP, 1080P, 30 FPS, full-color unit intended for use with a Raspberry-Pi V4 DPU. The smart mirror device 102 can include other cameras that are suitable for use with the DPU 104 and can obtain images of the patient and the medical device 112.

The DPU 104 of the smart mirror device 102 can perform computations and make determinations related to providing guidance for placing the medical device 112. For instance, the DPU 104 can make machine-learning model algorithmic and/or statistical decisions. The DPU 104 of the smart mirror device 102 can control the camera(s) 106 and the indicator(s) 108 of the smart mirror device 102. The DPU 104 can direct the camera(s) 106 to obtain one or more images of the patient. The DPU 104 can convey positioning information and other instructions to the patient. For instance, the DPU 104 can provide one or more of visual indicators and auditory indicators that direct the patient to move the medical device 112 to a target location, provide instructions to apply and use the medical device 112, and/or instruct the patient to pose in certain positions to obtain images. The DPU 104 can be a centralized device that includes all necessary software modules and databases to provide guidance to the patient. Optionally, the DPU 104 can be a distributed device that communicates with an external processor, such as the external processor 120. The DPU 104 may consist of a device, such as a Raspberry-Pi, embedded in the smart mirror device 102. The DPU 104 can be equipped with wired or wireless communications such as Wi-Fi, a cellular transceiver, or a Bluetooth LE (BLE) device.

The indicators 108 of the smart mirror device 102 can be any device suitable for providing guidance and/or instructions to the patient via the smart mirror device 102. For instance, the indicators 108 can be display screens, such as color graphics displays, that provide visual feedback to the patient. The indicators 108 may be illuminators, such as light emitting diodes (LEDs), that provide visual feedback to the patient. The indicators 108 may include speakers that provide auditory feedback to the patient.

The medical device 112 can include one or more sensors 116. For instance, the medical device 112 can include an integrated circuit magnetometer, accelerometer, and/or gyroscope that can provide data for determining the orientation of the medical device in three-dimensional space. The medical device 112 can be equipped with a wireless communication device for communicating with the smart mirror device 102. For example, the medical device 112 can include a Bluetooth device that can interconnect with a corresponding wireless communication device of the smart mirror device 102.

Optionally, the smart mirror device 102 can include one or more sensors, such as an integrated circuit magnetometer, accelerometer, and/or gyroscope. In one or more examples, the smart mirror device 102 can receive measurements from one or more of the sensors 116 of the medical device 112 and compare those measurements with measurements of the sensors of the smart mirror device 102 to obtain relative measurements. For instance, the smart mirror device 102 can determine the orientation of the medical device 112 relative to the smart mirror device 102 using measurements from sensors 116 of the medical device 112 and the sensors of the smart mirror device 102. In one or more examples, the sensors of the smart mirror device 102 can be used to measure and/or correct the orientation of the smart mirror device 102 when the smart mirror device 112 is mounted to a wall.

Optionally, the medical device 112 can include one or more indicators 118. For example, the indicators 118 can be light emitting diode (LED) indicators that inform the patient which direction to move the medical device 112 on the patient's body for optimal placement. Optionally, the indicators 118 may be tactile transducers for providing tactile feedback to the patient and/or speakers for proving audible feedback to the patient. In one or more examples, the medical device 112 can include one or more feedback devices that enable the patient to input information or confirmations that can be conveyed to the smart mirror device 102. For example, the medical device 112 can include a keypad or one or more buttons, or another input device that can receive information and/or feedback from the patient.

Optionally, the medical device 112 can include a DPU 114. The DPU 114 of the medical device 112 can be configured to convey positioning information and other instructions to the patient. For instance, the medical device 112 can include one or more indicators 118 that the DPU 114 can control in order to provide guidance to the patient as discussed above. The DPU 114 can be communicatively connected to a cloud-based processor and/or to the DPU 104 of the smart mirror device 102. For instance, the DPU 114 can be configured to communicate with the DPU 104 or a cloud-based processor via wireless communications such as Wi-Fi or Bluetooth LE.

The medical device 112 can be an on-body device such as a wearable drug delivery system. The wearable drug delivery system can include a reservoir holding a therapeutic agent, such as a reservoir of a container such as a vial, cartridge, or syringe. In one or more examples, the reservoir of the wearable drug delivery system must be within a specific orientation on the patient to successfully deliver the therapeutic agent. For instance, the wearable drug delivery system may rely on gravity to ensure that the therapeutic agent empties from the reservoir when it is administered to the patient. In such instance, it may be necessary for the patient to select a location for the wearable drug delivery system that ensures the delivery system is appropriately oriented, e.g., such that the reservoir is upright, while the patient uses the device. In addition to gravitational alignment, the medical device 112 may have other orientation restrictions. Exemplary restrictions can include preventing bubble formation in a reservoir, gravity-feed, valve orientation requirements, etc. Optionally, the medical device 112 can be an auto-injector or other fluid delivery apparatus such as a pre-filled syringe, pen injector, infusion set, catheter, on-body delivery device, or a combination thereof.

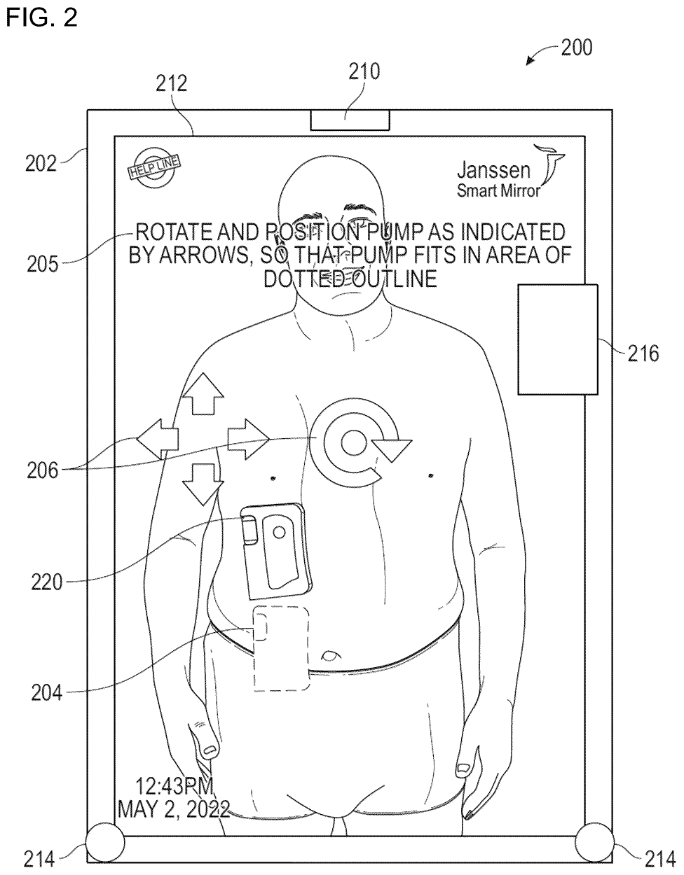

FIG. 2 shows an exemplary smart mirror system 200, according to one or more examples. As shown in FIG. 2, the smart mirror system 200 includes a smart mirror device 202 and a medical device 220. The smart mirror device 202 can include a variety of indicators including a display screen 212, speakers 214, and/or an illumination panel 216. Optionally, the smart mirror device 202 can include a DPU (not shown) that is embedded inside the smart mirror device 202. As shown, the smart mirror device 202 includes a mirror (showing a reflection of a patient) with embedded electronic devices (e.g., the camera 210, speakers 214, DPU). In one or more examples, the smart mirror device 202 can be a personal computer such as a laptop, tablet device, desktop PC, or smartphone that displays an image and/or video feed of the patient.

The display screen 212 can extend across any amount of the smart mirror device 202. As shown, the display screen 212 extends across substantially the entire surface of the smart mirror device 202. Optionally, the display screen 212 may extend across only a portion, such as 10%, 15%, 20%, 25%, or more of the surface of the smart mirror device 202. Optionally, the smart mirror device 202 can include a plurality of display screens 212. For instance, the smart mirror device 202 may include a first display screen that extends across a first portion of the smart mirror device 202, such as across the top portion, and one or more additional display screens located on other areas of the smart mirror device 202.

As shown in FIG. 2, the smart mirror device 202 includes two speakers 214 located on the lower corners of the smart mirror device 202. Optionally, the speakers 214 can be located in other areas of the smart mirror device 202, such as the top corners, the center, along the sides, or otherwise. The smart mirror device 202 can also include only one speaker 214, or more than two speakers 214.

The illumination panel 216 of the smart mirror device 202 can include a plurality of illuminators in a variety of shapes for providing guidance to the patient. For instance, the illumination panel 216 can include illuminators shaped to resemble objects such as arrows, so that when the illuminators are illuminated it is clear to the patient what the smart mirror device 202 is directing them to do. For instance, the illumination panel 216 can include a “down” arrow, such that when the “down” arrow is illuminated the patient understands they should move the medical device 220 downwards on their body. The illumination panel 216 can include light emitting diodes (LEDs), organic light emitting diodes (OLEDs), or other suitable illuminators. The illumination panel 216 can include illuminators configured to illuminate in a variety of colors.

Optionally, the smart mirror system 200 can be communicatively connected to an external device, such as the device of a health care provider. The display screen 212 of the smart mirror system 200 can include one or more feedback devices that allow the patient to enter information that can be communicated to the external device. For instance, the display screen 212 can include a touchscreen, an audio receiver for receiving voice commands, a physical or virtual keypad, etc.

As discussed above, the medical device 220 can include one or more indicators. The medical device 220 can include indicators configured to provide visual, auditory, or tactile feedback to the patient to provide guidance to the patient when locating the medical device. For example, the medical device 220 can include a motor or other device configured to vibrate with increased intensity the closer the patient moves the medical device 220 to the optimal location. The medical device 220 can include illuminators that flash as the patient moves the medical device 220 with increased frequency until the medical device 220 reaches the optimal location. The medical device 220 may include speakers that beep, alter frequency or pitch of a tone, or provide other guidance as the patient moves the medical device 220 to the optimal location. The guidance provided here is for example only and should not be construed as limiting. Other guidance could also be provided by indicators of the medical device.

As discussed above, the smart mirror system 200 can be configured to make machine-learning model algorithmic and/or statistical decisions. In one or more examples, the smart mirror system 200 can include a software system to determine the optimal location for placing a medical device on a patient's body based on images of the patient and to provide guidance to the patient to place the medical device in that location.

The smart mirror system 200 can provide guidance to the patient via one or more of the indicators of the smart mirror system 200. For example, the smart mirror system 200 can display one or more user interface objects on the display screen 212, emit auditory signals from the speakers 214, and/or provide guidance via illuminating one or more illuminators of the illumination panel 216. As shown in FIG. 2, the display screen 212 includes a text user interface object 205, which provides written instructions to the patient. The display screen 212 can also include arrow user interface objects 206, and a target user interface object 204.

In one or more examples, after determining the target location, the smart mirror device 202 can indicate this location via the target user interface object 204. After generating guidance to move the medical device 220 to the target location, the smart mirror device can provide that guidance to the patient via the display screen 212, such as by the arrow user interface objects 206, or via illuminators, such as by arrows of the illumination panel 216. As shown in FIG. 2, the target location for the medical device 220, indicated by the target user interface object 204, is located below the medical device 220. The smart mirror device 202 is thus indicating to the patient by the text user interface object 205 and the arrow user interface objects 206 how the patient should move and orient the medical device 220 to properly locate the medical device in the target location. These indications are provided for example only, and the smart mirror device 202 can provide guidance to the patient via other indicators of the smart mirror device 202 such as the speakers 214 and/or the illumination panel 216, and/or via indicators of the medical device 220 for placing the medical device in the target location.

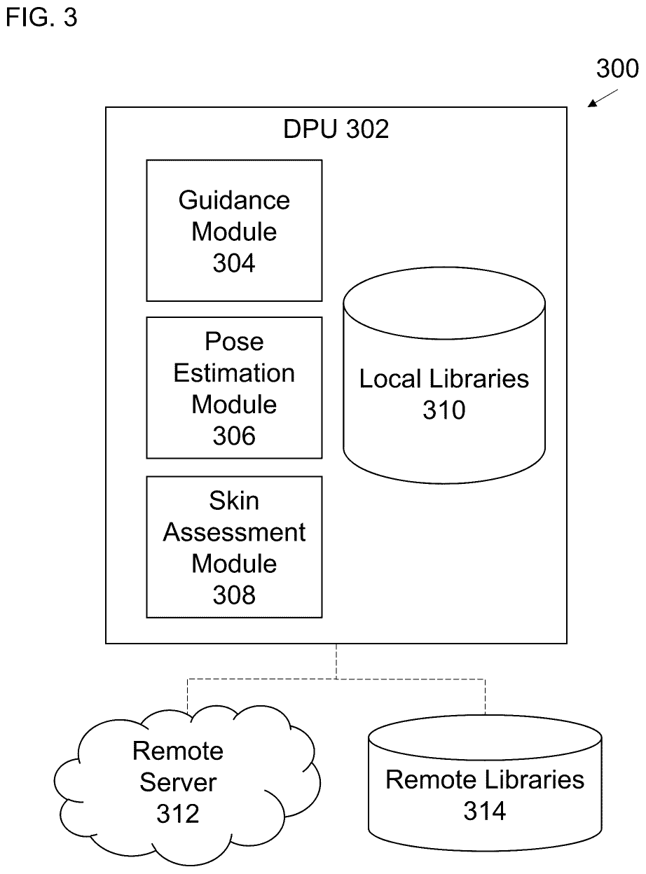

FIG. 3 shows an exemplary computing system 300 for providing guidance for placing a medical device to a patient via a smart mirror device, according to one or more examples. As shown, the computing system 300 can include a DPU 302 that includes one or more software modules and one or more local libraries 310. The DPU 302 can be embedded into a smart mirror device, such as the smart mirror system 200 or smart mirror device 102 described above.

The software modules of the DPU 302 may include a guidance module 304, a pose estimation module 306, and a skin assessment module 308. As will be described further below, the pose estimation module 306 can generate one or more body reference key points corresponding to body parts of the patient visible in images of the patient that the guidance module 304 can use to generate a photogrammetric model of the patient for determining the optimal placement of the medical device based on images of the patient.

The skin assessment module 308 can detect one or more dermatological conditions of the patient's skin and determine whether the skin is a suitable location for a medical device. For example, the skin assessment module 308 may detect that a patient's skin in a certain area is healthy and would be a suitable area for placing a medical device. The skin assessment module 308 may alternatively detect that a patient's skin in a certain area is damaged or exhibits some other condition and determine that the area is unsuitable for placing a medical device. Exemplary skin conditions that may be detected and determined to render an area unsuitable can include inflammation, scarring, infection, eczema, cancer, psoriasis, etc.

The skin assessment module 308 can detect dermatological conditions and make determinations based on those detected conditions by relying on a trained machine-learning (ML) model for detecting dermatological conditions. In one or more examples, the ML model for detecting dermatological conditions can be a convolutional neural network model.

In one or more examples, the skin assessment module 308, or other software modules of the DPU 302, may rely on historical data corresponding to the patient to determine whether a given area of the patient's body is suitable for placing the medical device. For instance, the skin assessment module 308 can consult a historical database that contains information regarding the previous locations where the patient has placed a medical device on their body. If the data in the historical database indicates that the patient last administered a medical device in a particular area or location within a specified time period, the skin assessment module 308 may classify the area or location as unsuitable. For example, if the patient administered a medical device in a particular area or location within one week, that area or location may be deemed unsuitable because that area or location may present difficulties with puncturing the patient's skin and/or adhering the device to the skin, and to avoid causing repeated damage to the patient's skin in that area. The period of time after a given dose that renders the skin unsuitable for another injection may vary based on the medical device and/or the therapeutic agent being administered. An exemplary period where a new dose should not be administered is within one week of a previous dose. Alternatively, this period may be in the span of hours, such as within 1, 2, 3, 4, or 5 hours of the last dose. The period may be on the span of days, such as within 1, 2, 3, 4, or 5 days of the last dose. Alternatively, the period may be on the span of weeks or months within the last dose.

In one or more examples, the guidance module 304 can guide the patient through the process of preparing, optimally locating, applying, and/or administering the medical device. As will be described in depth below, the guidance module can determine a target location for placing the medical device based on the information and determinations made by the skin assessment module 308 and/or the pose estimation module 306. The guidance module 304 can generate guidance for moving the medical device to the target location. The guidance module 304 can then provide this guidance to the patient via one or more indicators of the smart mirror device, such as the display, speakers, and/or illuminators of the smart mirror device discussed above.

The local libraries 310 can store software and/or any number of databases of information used by the software modules of the DPU 302. Optionally, the DPU 302 may be communicatively connected to a remote server 312 and/or one or more remote libraries 314. When in communication with a remote server 312, the DPU 302 may relay information to the remote server 312 to perform complex calculations or to perform one or more of the detection and/or determination decisions that are used by the guidance module 304 when generating and providing guidance to a patient for placing a medical device. The remote libraries 314 can store software and/or information used for the detection and/or determination systems of the guidance module 304.

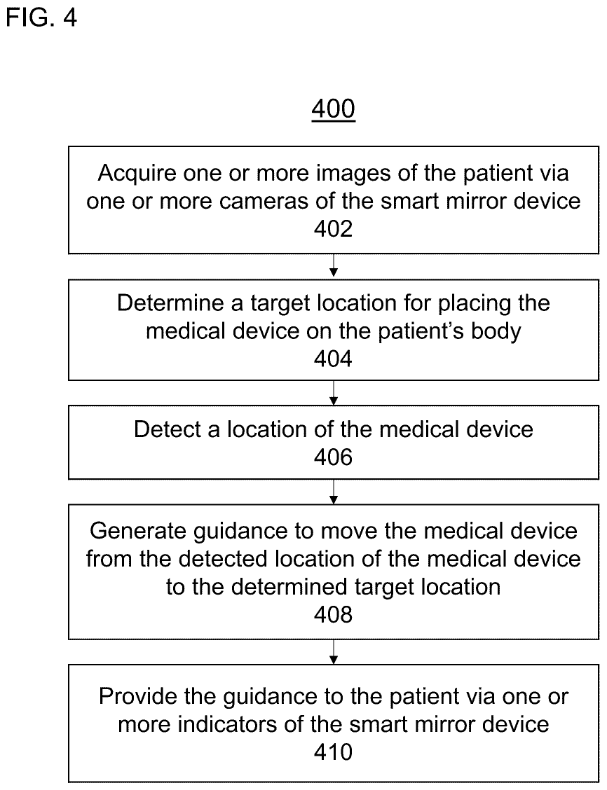

FIG. 4 shows an exemplary method 400 for providing guidance for placing a medical device to a patient via a smart mirror device, according to one or more examples. The method 400 can be performed by a DPU of a smart mirror device, such as the smart mirror devices discussed above. In one or more examples, the method 400 can begin at step 402 with acquiring one or more images of the patient via one or more cameras of a smart mirror device. As discussed above, the smart mirror device can include one or more cameras and a DPU with a controller. Acquiring one or more images of the patient at step 402 can involve the DPU controlling the one or more cameras of the smart mirror device to capture images of the patient. In one or more examples, the one or more images can be acquired from a video of the patient, with the individual images extracted from the video.

In one or more examples, acquiring the one or more images of the patient at step 402 can involve the smart mirror device providing instructions to the patient regarding how to pose for the images and directing the patient to alter their pose such that the one or more images can be used to determine a target location for placing a medical device on the patient's body. The instructions can be provided by a guidance software module, such as the guidance module 304 of the DPU 302 of FIG. 3. The instructions can be provided via indicators of the smart mirror device. For instance, the guidance can be provided in the form of auditory instructions emitted from speakers, visual icons or images displayed on a display of the smart mirror device, or via illuminated illuminators.

The smart mirror device can provide instructions to the patient to pose for a reference photo, which can include directing the patient to stand directly in front of a camera of the smart mirror device with their arms outstretched on either side of their body such that their body forms a “T” shape. The instructions provided can also include instructing the patient to wear certain clothing, such as tight-fitting clothing or minimal clothing such that the images obtained can clearly delineate the patient's body shape. The instructions can include directing the patient to pose for a variety of supplemental images, and/or directing the patient to orient their body in a variety of configurations relative to the camera of the smart mirror device so that the images clearly depict the shape of the patient's body.

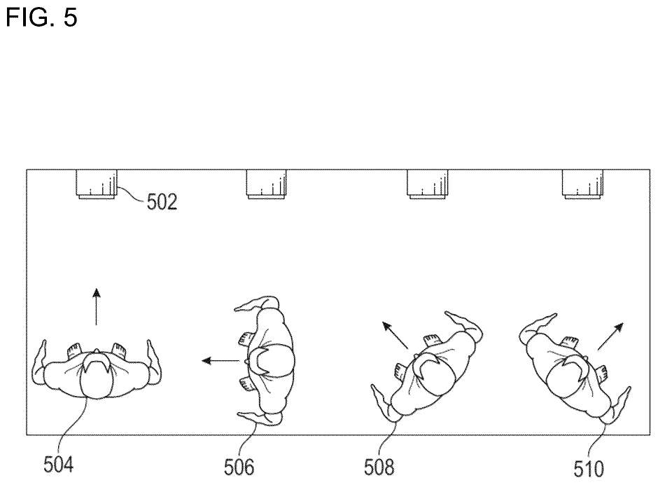

FIG. 5 shows an exemplary depiction of a reference image 504 and supplemental images 506, 508, 510 of a patient that can be obtained by a smart mirror device system, according to one or more examples. As shown in FIG. 5, in the reference image 504, the patient is directly facing the camera 502. In the supplemental images 506, 508, and 510, however, the patient is standing in a variety of orientations relative to the camera 502. In one or more examples, when providing instructions to acquire the images of the patient at step 402, the smart mirror device can instruct the patient to stand directly perpendicular to the camera 502 as depicted in the supplemental image 506. The smart mirror device can also provide guidance to the patient to stand diagonally relative to the camera 502, as shown in the supplemental images 508 and 510. Optionally, the smart mirror device will instruct the patient to stand with his or her feet slightly apart with unbent arms raised outward from his or her body by 45 to 90 degrees such that their outstretched arms for a “T” (hereafter, the “T-pose). In one or more examples, the smart mirror device can instruct the patient to stand in the T-pose and to rotate 360 degrees in a slow, continuous manner, and obtain a continuous set of one or more images or a video.

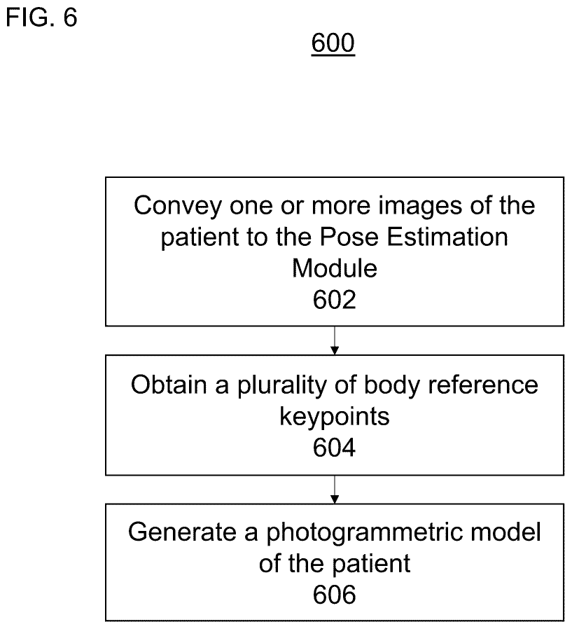

After acquiring the one or more images at step 402, the method 400 can move to step 404 and determine a target location for placing the medical device on the patient's body. In one or more examples, determining the target location at step 404 can include generating a photogrammetric model of the patient and identifying specific areas as unsuitable based in part on the photogrammetric model. FIG. 6 shows an exemplary method 600 for generating a photogrammetric model of a patient, according to one or more examples. The method 600 can be executed by a DPU of a smart mirror device, such as the DPUs discussed above, as part of the method 400 for providing guidance for placing a medical device to a patient via the smart mirror device. In one or more examples, the method 600 can begin at step 602 with conveying one or more images of the patient to a software module for estimating pose. The images being conveyed at step 602 can be the images of the patient acquired at step 402 of the method 400.

Conveying the one or more images at step 602 can involve conveying the images to the pose estimation module 306 of the DPU 302 referenced above. In one or more examples, the pose estimation module can be stored externally from the DPU of the smart mirror device, and conveying the one or more images at step 602 can involve transmitting the images via wireless communication to the external pose estimation module. In one or more examples, the pose estimation module can be a trained ML model for estimating pose. Optionally, the pose estimation module can be a convolutional neural network model.

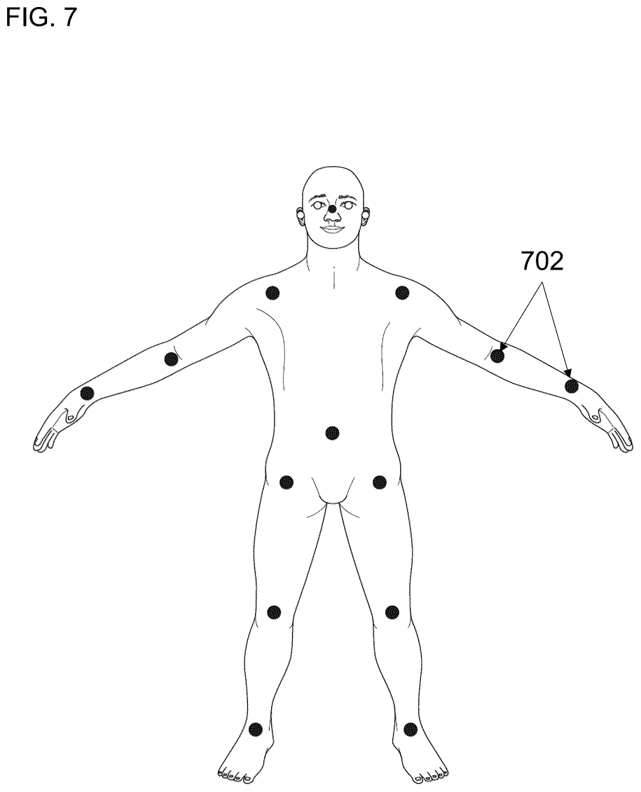

After conveying the one or more images to the pose estimation module at step 602, the method 600 can move to step 604 and obtain a plurality of body reference keypoints. The body reference keypoints can be obtained from the pose estimation module. The pose estimation module can be configured to receive a number of images of a person and generate a number of body reference keypoints of the person based on those images. An exemplary output of a pose estimation module is shown in FIG. 7, which shows exemplary extracted body reference keypoints 702 of a person's body 700 for use in providing guidance to place a medical device, according to one or more examples. As shown in FIG. 7, there are eighteen distinct body reference keypoints 702 corresponding to distinct points of the person's body 700. For instance, there are keypoints for a left and right wrist, elbow, shoulder, hip, knee, ankle, eye, and ear, as well as keypoints for a nose and a navel. In one or more examples, the body reference keypoints obtained at step 604 can be obtained in near real-time after obtaining the reference image and the plurality of supplemental images of the patient.

Referring now back to FIG. 6, after obtaining the body reference keypoints at step 604, the method 600 can move to step 606 and generate a photogrammetric model of the patient. The photogrammetric model generated at step 606 can be based on the one or more images of the patient. The photogrammetric model can be a three dimensional model of the patient's body. If a patient's body is curved, for instance curved outwardly at their midsection, the photogrammetric model can illustrate the curves of the patient's body. In one or more examples, the photogrammetric model generated via method 600 can be used to generate custom guidance for placing the medical device on the patient's body that takes into account the patient's curvature, ensuring that the device can be adequately adhered and applied when located in the target location. After generating the photogrammetric model via method 600, the model can be stored locally on the DPU of the smart mirror device, or saved to an external database communicatively connected to the smart mirror device.

Referring now back to FIG. 4, determining a target location at step 404 of the method 400 can include identifying unsuitable areas of the patient's body. In one or more examples, identifying the unsuitable areas can be based in in part on orientation limitations of the medical device and the patient's curvature as evidenced by the photogrammetric model. For instance, certain medical devices may require that the device be in a specific orientation, such as aligned with the gravitational vertical, to successfully deliver the therapeutic agent. Accordingly, the patient's body in a particular area may be curved such that attempting to apply and administer the medical device in that area would not result in a proper drug delivery.

Identifying the one or more unsuitable areas of the patient's body based on the photogrammetric model can include determining various vectors based on the photogrammetric model in a particular area and determining whether that area is a suitable location for applying the medical device based on those vectors. For instance, the smart mirror device can determine three-dimensional normal and/or gradient surface vectors for the particular area of the patient's body using the photogrammetric model. The smart mirror device can then compare those normal and/or gradient surface vectors to the orientation limitations of the medical device and determine whether the area is a suitable target location for the medical device.

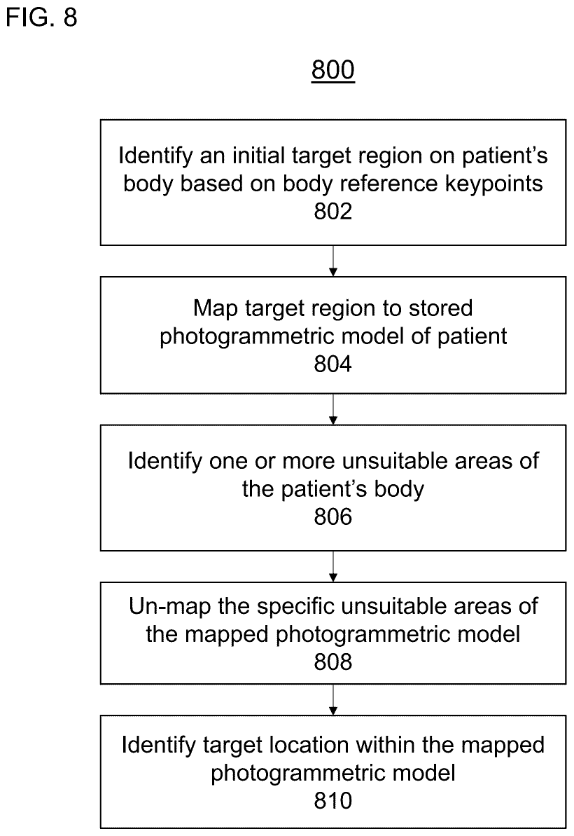

FIG. 8 shows an exemplary method 800 for identifying a target location for placing a medical device. In one or more examples, the method 800 can be executed by the DPU of a smart mirror device while performing step 404 of the method 400. The method 800 can begin at step 802 with identifying an initial target region on the patient's body based on one or more body reference keypoints. The initial target region at step 802 can be identified based in part on the body reference keypoints of the patient. For example, the initial target region can be confined to an area of the patient's midsection, as identified based on the body reference keypoints. In one or more examples, the initial target region can be selected based in part on protocol specific to the medical device. For instance, a given medical device may contain standard protocol that dictates the optimal target location is aligned with the patient's navel and located a specified distance, such as less than 10 inches, from the navel. The initial target region identified at step 802 can be a region that surrounds this location.

After identifying the initial target region at step 804, the method 800 can move to step 804 and map the target region to the stored photogrammetric model of the patient. As discussed above, a photogrammetric model can be generated and stored that illustrates a three-dimensional rendering of the patient's curvature. Mapping the target region to the stored photogrammetric model at step 804 can involve excerpting only the portion of the photogrammetric model that corresponds to the target region.

After mapping the initial target region at step 804, the method 800 can move to step 806 and identify one or more unsuitable areas of the patient's body. As discussed above, the one or more unsuitable areas can include areas of the patient's body with one or more of inflammation, scarring, infection, eczema, cancer, psoriasis, skin tags, moles, lipomas, cysts, etc. The one or more unsuitable areas can include areas deemed unsuitable based in part on how much time has elapsed since the patient last administered the medical device in those areas. The one or more unsuitable areas can include areas determined to be unsuitable for applying the medical device based on a photogrammetric model of the patient in a particular area. These unsuitable areas can be detected by one or more software modules, as discussed above. Identifying the one or more unsuitable areas at step 806 may be confined only to analyzing the region of the patient's body in the initial target region, to minimize the computational processing required (e.g., to minimize the area wherein unsuitable areas are detected). In one or more examples, while identifying unsuitable areas at step 808, the patient may be directed to re-pose and additional photos may be acquired of a potential target area, with those additional photos scrutinized to identify any unsuitable areas.

After identifying the one or more unsuitable areas at step 806, the method 800 can move to step 808 and un-map the one or more unsuitable areas from the mapped photogrammetric model. Un-mapping the unsuitable areas from the mapped photogrammetric model at step 808 can involve remove the areas determined to be unsuitable such that the only portions of the photogrammetric model that are “mapped” are those areas within the target region that were not deemed to be unsuitable.

After un-mapping the one or more unsuitable areas at step 808, the method 800 can move to step 810 and identify a target location within the mapped photogrammetric model. Identifying the target location within the mapped region of the photogrammetric model at step 810 can involve calculating a point (via a DPU of the smart mirror device) in the mapped region that is closest to a centroid of the mapped region. Optionally, identifying the target location within the mapped region of the photogrammetric model at step 810 can involve selecting a pseudorandom point on the mapped region. Identifying the target location within the mapped region of the photogrammetric model at step 810 can involve selecting a point on the mapped region according to a predefined algorithm. One exemplary algorithm for identifying the target location within the mapped region can include sub-dividing the target region into N polygons of equal area and then examining the centroid of each polygon consecutively.

Returning back to FIG. 4, after determining the target location at step 404, the method 400 can move to step 406 and detect a location of the medical device. In one or more examples, detecting the location of the medical device at step 406 can involve identifying one or more device reference keypoints based on the one or more images of the patient obtained at step 402 of the method 400. The smart mirror device may convey instructions to the patient when acquiring the images at step 402 that the patient should hold the medical device in the image so that the device can be detected by the smart mirror device. The DPU may detect the medical device using a machine-learning model trained to detect certain medical devices. In one or more examples, the software and/or machine-learning model for detecting medical devices can be stored and executed locally by the DPU of the smart mirror device. Alternatively, the software and/or the machine-learning model can be stored and executed on an external server with the results communicated back to the smart mirror device.

In one or more examples, detecting the location of the medical device at step 406 can be based on the body reference keypoints generated based on the one or more images of the patient. For instance, the DPU of smart mirror device may detect that the patient is holding the medical device in their right hand in the one or more images of the patient and determine the location of the medical device based on that determination. Detecting the location of the medical device at step 406 can involve extracting a custom set of anthropometric ratios for the patient based in part on the body reference keypoints. The anthropometric ratios extracted can be refined in accordance with one or more of patient race, gender, age, and/or other categorical variations.

After extracting the custom set of anthropometric ratios for the patient, the smart mirror device can use that information to infer a location of the medical device. For example, the smart mirror device may detect that the patient is a 70^{th} percentile-sized woman and access a database containing anthropometric data for a human of that size to determine the measurements of the patient's anatomy to use to infer the location of the medical device. The smart mirror device may use this information in conjunction with other software that can detect the angles at which portions of the patient's arms are arranged to determine where the medical device is located relative to other areas of the patient's body. Optionally, the smart mirror device extracts custom anthropometric ratios from images of the patient (such as via an ML program) and uses those custom anthropometric ratios to determine the location of the medical device.

In one or more examples, detecting the location of the medical device at step 406 of the method 400 can be based in part on input from one or more sensors of the medical device. As discussed above, the medical device can include sensors such as a magnetometer, an accelerometer, and/or gyroscope that provide data for determining the orientation of the medical device in three-dimensional space. This orientation information may be used when detecting the location of the medical device. For instance, the smart mirror device may determine that the medical device is off-set from the vertical (as shown by the medical device 220 of FIG. 2) and use that information to determine the relative angles of portions of the patient's body to infer the location of the medical device. Optionally, detecting the location of the medical device at step 406 can be based in part on a determination as to how far the medical device is from a receiver on the smart mirror device.

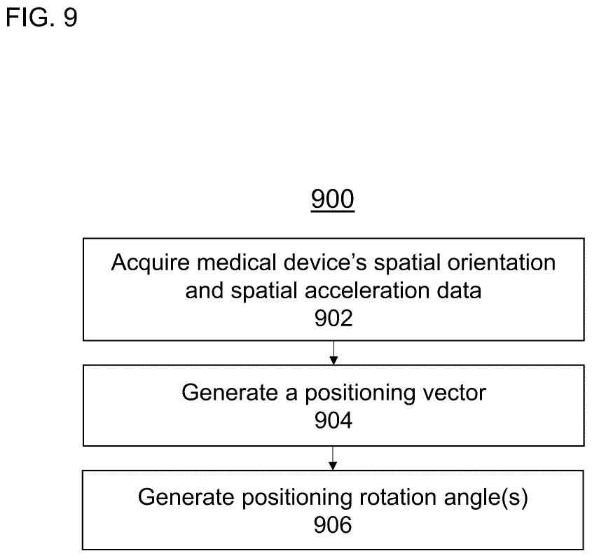

After detecting the location of the medical device at step 406, the method 400 can move to step 408 and generate guidance to move the medical device from the detected location of the medical device to the determined target location. FIG. 9 shows an exemplary method 900 for generating positioning guidance for placing a medical device. According to one or more examples, the method 900 can be executed by a software module, such as by the guidance module 304 of the DPU 302 of FIG. 3, while generating guidance at step 408 of the method 400.

The method 900 can begin at step 902 with acquiring the medical device's spatial orientation and spatial acceleration data. As discussed above, the medical device can include sensors such as a magnetometer, an accelerometer, and/or gyroscope that provide data for determining the orientation of the medical device in three-dimensional space. Acquiring the medical device's spatial orientation and acceleration data at step 902 of the method 900 can involve the smart mirror device communicating with the medical device to acquire this information. For instance, the medical device can obtain readings from the sensors of the medical device in response to a request from the smart mirror device for the information, and then convey that information to the smart mirror device.

After acquiring the medical device's spatial orientation and acceleration data at step 902, the method 900 can move to step 904 and generate a positioning vector. The positioning vector generated at step 904 can be a two-dimensional vector that can be used for positioning guidance. In one or more examples, the 2D positional vector can be based on the detected device reference keypoints. Generating the 2D positional vector based on the detected device reference keypoints can involve subtracting coordinates based on the device reference keypoints from the coordinates of the target position.

Optionally, the 2D positional vector can be generated based on the body reference keypoints of the patient and the custom set of anthropometric ratios for the patient. The smart mirror device can infer a set of device reference keypoints after inferring the position of the medical device. For example, if the smart mirror device detects that the patient is holding the medical device in their right hand, the smart mirror device can infer that the medical device location lies along the vector defined by the right-elbow to right-wrist keypoints of the patient's body reference keypoints, and further infer that the medical device is located away from the right wrist keypoint defined by the standard anthropometric ratio of the radius bone length to the wrist/median palmer length. The 2D positional vector can then be generated based on this inferred location, by subtracting coordinates corresponding to the inferred device location from the coordinates of the target position.

After generating the positioning vector at step 904, the method 900 can move to step 906 and generate one or more positioning rotation angle(s). The positioning rotation angles generated at step 906 can be three-dimensional vectors that can be used for positioning guidance. In one or more examples, while generating the positioning vector at step 904, the method 900 can include providing instructions to the patient to adjust the orientation of the medical device (such as to hold the device such that its face is parallel to the mirror) before/while generating the positioning vectors.

In one or more examples, the 3D positioning rotation angle generated can be based on the detected device reference keypoints. For example, if the smart mirror device detected two or more device reference keypoints, the smart mirror device can determine the medical device's vertical orientation vector (e.g., the device's “up” direction) and then calculate the dot product of this vector and the local gravitation vector (as determined by the camera vertical direction or by the symmetry axis of the patient's coordinates). The device's vertical orientation vector and the local gravitation vector can be used to generate the positioning rotation angle, such as by subtracting the device's vertical orientation from the local gravitation vector. The positioning rotation angle can be conveyed to the patient as a positioning rotation direction, for instance by indicating the patient should tilt the device to the right or left, as will be discussed below.

In one or more examples, the 3D positioning rotation angle generated can be based on the medical device's spatial orientation and spatial acceleration data acquired at step 902 of the method 900. The medical device can include an accelerometer, which can be used to determine the medical device's alignment relative to the gravitational vertical. Optionally, the 3D positioning rotation angle generated can be based on both the detected device reference keypoints and the medical device's spatial orientation and spatial acceleration data.

Returning now back to FIG. 4, after generating guidance at step 408, the method 400 can move to step 410 and provide the guidance to the patient via the one or more indicators of the smart mirror device. As discussed above and as illustrated in FIG. 2, the indicators of the smart mirror device can include a display screen 212, one or more speakers 214, and an illumination panel 216.

Providing guidance to the patient at step 410 of the method 400 can include displaying one or more user interface objects on a display of the smart mirror device as discussed above. For instance, providing guidance can include displaying one or more user interface objects corresponding to the 2D positioning vector and/or 3D positioning vectors generated at step 408 of the method 400. Exemplary user interface objects corresponding to 2D and 3D positioning vectors are shown in FIG. 2. For example, the display screen 212 of the smart mirror system 200 includes arrow user interface objects 206 which can corresponding to the 2D and 3D positioning vectors generated at step 408 of the method 400. The target user interface object 204 and the text user interface object 205 of the display screen 212 can also correspond to the guidance generated at step 408 of the method 400 and the target location determined at step 404 of the method 400.

Providing guidance to the patient at step 410 of the method 400 can include emitting auditory guidance from the speakers 214 of the smart mirror device. For example, the smart mirror device may emit the pre-recorded voice of a health care provider who is instructing the patient to move the medical device according to the guidance generated at step 408 off the method 400. The smart mirror device may emit the voice of a health care provider who is instructing the patient to move the medical device in real-time, with the smart mirror device communicatively connected to a device of the health care provider.

Providing guidance to the patient at step 410 of the method 400 can include illuminating one or more illuminators of the illumination panel 216. For example, the illumination panel may include illuminators shaped to resemble one or more arrows, and providing guidance can include illuminating the illuminators corresponding to the guidance generated at step 408 of the method 400, such as by illuminating the “down” arrow to indicate the patient should move the patient downwards on their body. In one or more examples, providing guidance to the patient at step 410 can involve relying on a combination of displaying user interface objects on a display screen, emitting auditory guidance via speakers, and illuminating illuminators of the smart mirror device. Additionally or alternatively, providing guidance to the patient at step 410 can involve emitting guidance via one or more indicators of the medical device as discussed above.

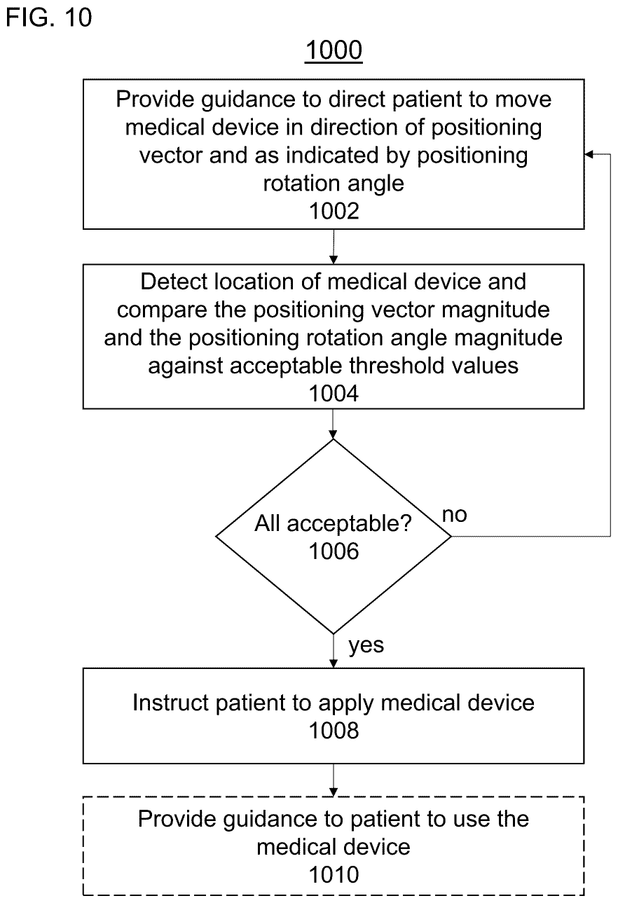

FIG. 10 shows an exemplary method 1000 for providing positioning guidance for placing a medical device. According to one or more examples, the method 1000 can be executed by a DPU of a smart mirror device, such as the DPUs discussed above, as part of the method 400 for providing guidance for placing a medical device to a patient via the smart mirror device. In one or more examples, the method 1000 can begin at step 1002 with providing guidance to direct the patient to move the medical device in the direction of a positioning vector and as indicated by the positioning rotation angle. Providing guidance at step 1002 can involve providing guidance via any of the indicators discussed above, such as by displaying user interface objects on a smart mirror device, illuminating illuminators of the smart mirror device, emitting auditory guidance from the smart mirror device.

After providing guidance at step 1002, the method 1000 can move to step 1004 and detect the location of the medical device and compare that to the target location. The smart mirror device may continuously detect the location of the medical device as the patient moves the medical device. Optionally, the smart mirror device may detect the location of the medical device incrementally after providing guidance to the patient. For instance, the smart mirror device may detect the location of the medical device immediately after providing guidance and then repeatedly detect the location after a specified period has elapsed after providing guidance, such as once each second after providing guidance. Detecting the location of the medical device at step 1004 can be performed by any of the methods described above, such as by locating device reference keypoints and/or relying on a custom set of anthropometric ratios for the patient.

At step 1006, the method 1000 can determine whether the location of the medical device is acceptable, e.g., whether the location of the medical device matches the target location within an acceptable margin of error. If the medical device location does not match the target location, the method 1000 may return to step 1002 and continue to provide guidance to the patient. If the medical device location does match the target location, the method 1000 can move to step 1008 and instruct the patient to apply the medical device. In one or more examples, the smart mirror device may notify the patient via one or more of the indicators of the smart mirror device and/or the medical device that the medical device is in the target location. For example, the smart mirror device may emit a beeping sound from one or more speakers and/or display a checkmark user interface object on the display screen of the smart mirror device.

In one or more examples, step 1006 of the method 1000 can involve continually comparing the magnitude of the positioning vector and the positioning rotation angle of the medical device to predetermined maximum thresholds to determine whether the positioning vector and the positioning rotation angle of the medical device are collectively small enough to determine the medical device placement is appropriate. If the vectors are not acceptable, indicating that the patient has not yet moved the medical device all the way to the target location (or not within an acceptable margin of error of the target location), the method 1000 may return to step 1002 and continue to provide guidance to the patient.

Instructing the patient to apply the medical device at 1008 can include providing instructions to the patient by one or more indicators of the smart mirror device and/or the medical device. For example, the smart mirror device may provide instructions in the form of text user interface objects displayed on the display screen or a recording of a healthcare provider via speakers of the smart mirror device that indicate how the patient should adhere the medical device to their body.

Optionally, after instructing the patient to apply the medical device at step 1008, the method 1000 can include step 1010 and instruct the patient to use the medical device. Instructing the patient to use the medical device at step 1010 can involve providing instructions to the patient regarding how to administer a therapeutic agent of the medical device, how the patient should pose their body during the administration of the therapeutic agent (such as remain standing or lie down), what to do when the administration is complete, how to dispose of the medical device thereafter, etc. Instructing the patient to use the medical device at step 1010 can involve displaying user interface objects on a display screen of the smart mirror device, emitting auditory guidance, illuminating illuminators of the smart mirror device, providing feedback from an indicator of the medical device, etc.

In one or more examples, instructing the patient to use the medical device at step 1010 can involve monitoring the orientation of the medical device to ensure the medical device remains in an appropriate orientation relative to orientation limitations of the medical device. For example, if the medical device must remain upright to successfully deliver a therapeutic agent, monitoring the orientation of the medical device may include acquiring the medical device's spatial orientation data, such as by a magnetometer, an accelerometer, and/or gyroscope of the medical device or based on detecting the medical device in images of the patient obtained after the patient began administering the medical device, and using that information to determine whether the medical device remains upright.

Optionally, the smart mirror device may monitor the orientation of the medical device at discrete intervals after the patient begins administering the medical device. For example, the smart mirror device may obtain information regarding the orientation of the medical device every 10 seconds, 20 seconds, 1 minute, 2 minutes, etc. after the patient begins administering the medical device. The monitoring intervals may be based on the IFU of the particular medical device. For instance, a medical device that requires 10 minutes total for administration may be monitored more frequently than a medical device that requires 1 hour total for administration. If while monitoring the orientation of the medical device the smart mirror device determines that the orientation is not appropriate, the smart mirror device may provide guidance to the patient, such as by flashing lights on the smart mirror device and/or medical device or displaying user interface objects, etc. on the smart mirror device to guide the patient to fix the orientation.

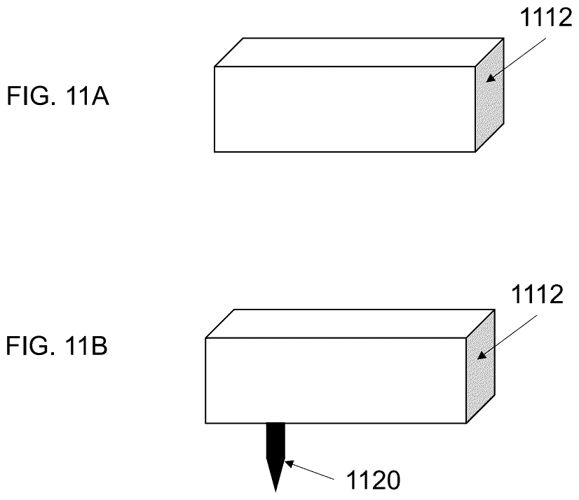

FIG. 11A illustrates a side view of an exemplary auto-injector medical device 1112, and FIG. 11B illustrates a side view of the exemplary medical device 1112 of FIG. 11A with the needle 1120 protruding from the body of the medical device 1112. When a patient uses the medical device 1112, the backside of the device (e.g., the side from which the needle 1120 protrudes while in use) contacts the patient's body. When placing the medical device 1112, however, the needle 1120 is located within the medical device 1112 (as shown in FIG. 11A). Accordingly, the patient may not know where the needle 1120 is located, or will be located once it protrudes, when placing the medical device 1112 on their body. Similar to the medical device 112 of FIG. 1, the medical device 1112 can include one or more sensors, a communication device, and a DPU for communicating to a smart mirror device, such as the smart mirror device 102 of FIG. 1. The medical device 1112 can also include one more indicators, such as one or more LEDs, speakers, and/or tactile transducers, for providing directions to the patient when placing the medical device 1112 on their body.

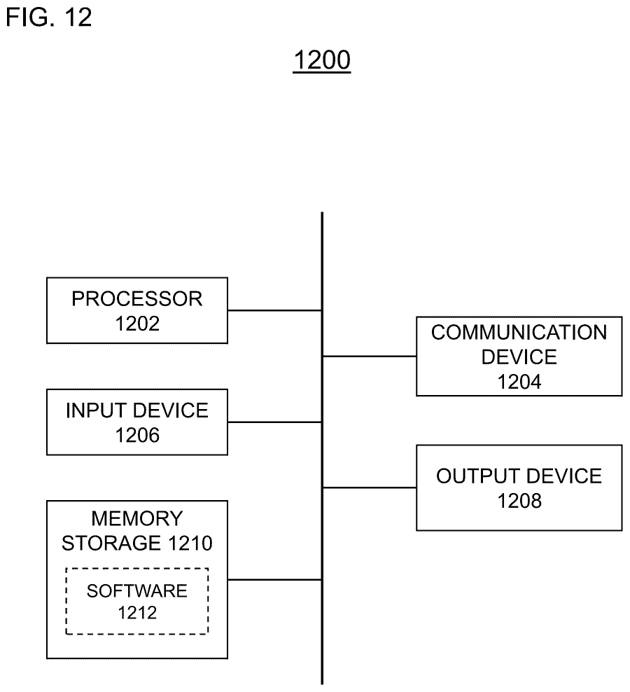

FIG. 12 illustrates an example of a computing device 1200, according to one or more examples. In one or more examples, the DPU of the smart mirror device 102 of FIG. 1 can include the computing device 1200, such as in place of the DPU 104. In one or more examples, the external processor 120 of FIG. 1 can include the computing device 1200. Device 1200 can be a host computer connected to a network. Device 1200 can be a client computer or a server. As shown in FIG. 12 device 1200 can be any suitable type of microprocessor-based device, such as a personal computer, workstation, server, or handheld computing device (portable electronic device) such as a phone or tablet. The device can include, for example, one or more of processors 1202, input device 1206, output device 1208, storage 1210, and communication device 1204. Input device 1206 and output device 1208 can generally correspond to those described above and can either be connectable or integrated with the computer.

Input device 1206 can be any suitable device that provides input, such as a touch screen, keyboard or keypad, mouse, or voice-recognition device. Output device 1208 can be any suitable device that provides output, such as a touch screen, haptics device, or speaker.

Storage 1210 can be any suitable device that provides storage, such as an electrical, magnetic, or optical memory, including a RAM, cache, hard drive, or removable storage disk. Communication device 1204 can include any suitable device capable of transmitting and receiving signals over a network, such as a network interface chip or device. The components of the computer can be connected in any suitable manner, such as via a physical bus or wirelessly.

Software 1212, which can be stored in storage 1210 and executed by processor 1202, can include, for example, the programming that embodies the functionality of the present disclosure (e.g., as embodied in the devices as described above).

Software 1212 can also be stored and/or transported within any non-transitory computer-readable storage medium for use by or in connection with an instruction execution system, apparatus, or device, such as those described above, that can fetch instructions associated with the software from the instruction execution system, apparatus, or device and execute the instructions. In the context of this disclosure, a computer-readable storage medium can be any medium, such as storage 1210, that can contain or store programming for use by or in connection with an instruction execution system, apparatus, or device.

Software 1212 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate, or transport programming for use by or in connection with an instruction execution system, apparatus, or device. The transport readable medium can include, but is not limited to, an electronic, magnetic, optical, electromagnetic, or infrared wired or wireless propagation medium.

Device 1200 may be connected to a network, which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 1200 can implement any operating system suitable for operating on the network. Software 1212 can be written in any suitable programming language, such as C, C++, Java, or Python. In various embodiments, application software embodying the functionality of the present disclosure can be deployed in different configurations, such as in a client/server arrangement or through a Web browser as a Web-based application or Web service, for example.

The foregoing description, for the purpose of explanation, has been described with reference to specific examples. However, the illustrative discussions above are not intended to be exhaustive or to limit the invention to the precise forms disclosed. Many modifications and variations are possible in view of the above teachings. The examples were chosen and described in order to best explain the principles of the techniques and their practical applications. Others skilled in the art are thereby enabled to best utilize the techniques and various examples with various modifications as are suited to the particular use contemplated.

Although the disclosure and examples have been fully described with reference to the accompanying figures, it is to be noted that various changes and modifications will become apparent to those skilled in the art. Such changes and modifications are to be understood as being included within the scope of the disclosure and examples as defined by the claims.

1. A method for guiding a patient to place a medical device on the patient's body using a computing device, the method comprising:
acquiring one or more images of the patient via one or more cameras of the computing device;
detecting one or more characteristics of the patient's body in the one or more images;
determining a target location for placing the medical device on the patient's body based on the detected one or more characteristics;
detecting a location of the medical device;
generating guidance for moving the medical device from the detected location of the medical device to the determined target location; and
providing the guidance to the patient via one or more indicators of the computing device.

2. The method of claim 1, wherein the computing device comprises a mirror.

3. (canceled)

4. (canceled)

5. (canceled)

6. (canceled)

7. The method of claim 1, wherein determining the target location comprises identifying one or more unsuitable areas of the patient's body based on the detected one or more characteristics.

8. The method of claim 7, wherein (i) the one or more unsuitable areas of the patient's body comprise areas with one or more of inflammation, infection, eczema, cancer, and psoriasis and/or (ii) identifying the one or more unsuitable areas is based in part on how much time has elapsed since the patient last administered an injection from any medical device, and/or (iii) identifying the one or more unsuitable areas is based in part on orientation limitations of the medical device.

9. (canceled)

10. (canceled)

11. The method of claim 7, wherein determining the target location comprises generating a photogrammetric model of the patient and identifying the one or more unsuitable areas is based in part on the photogrammetric model.

12. The method of claim 11, wherein identifying the one or more unsuitable areas comprises determining three dimensional normal surface vectors of the photogrammetric model, and the one or more unsuitable areas comprise areas wherein the three dimensional normal surface vectors are incompatible with one or more orientation limitations of the medical device.

13. The method of claim 11, wherein identifying the one or more unsuitable areas comprises determining three dimensional gradient surface vectors of the photogrammetric model, and the one or more unsuitable areas comprise areas wherein the three dimensional gradient surface vectors are incompatible with one or more orientation limitations of the medical device.

14. The method of claim 11, wherein generating the photogrammetric model comprises obtaining a plurality of body reference keypoints in near real-time based on the one or more images.

15. The method of claim 14, wherein determining the target location comprises identifying a target region on the patient's body based on the body reference keypoints and identifying the target location within the target region based on the one or more unsuitable areas.

16. The method of claim 15, wherein determining the target location comprises:
mapping the target region to the photogrammetric model of the patient;
un-mapping the one or more unsuitable areas from the mapped photogrammetric model; and
identifying the target location within the mapped photogrammetric model.

17. (canceled)

18. (canceled)

19. The method of claim 14, wherein obtaining the plurality of estimated body reference keypoints comprises processing the one or more images using a machine-learning model for estimating pose.

20. The method of claim 19, wherein the machine-learning model is a convolutional neural network model.

21. The method of claim 14, wherein detecting the location of the medical device comprises extracting a custom set of anthropometric ratios for the patient based in part on the body reference keypoints and inferring the location of the medical device based on the custom set of anthropometric ratios for the patient.

22. The method of claim 21, wherein generating guidance comprises generating a 2D positioning vector based on the body reference keypoints and the custom set of anthropometric ratios for the patient, and wherein the one or more user interface objects comprise a user interface object corresponding to the 2D positioning vector.

23. The method of claim 1, wherein detecting the location of the medical device comprises identifying one or more device reference keypoints based on the one or more images.

24. The method of claim 23, wherein generating guidance comprises generating a 2-dimensional (2D) positioning vector based on the one or more device reference keypoints and the target location, and wherein the one or more user interface objects comprise a user interface object corresponding to the 2D positioning vector on the computing device.

25. The method of claim 24, wherein generating guidance comprises generating a 3-dimensional (3D) positioning rotation angle based on the one or more device reference keypoints and the target location, and wherein the one or more user interface objects comprise a user interface object corresponding to the 3D positioning rotation angle on the computing device.

26. (canceled)

27. The method of claim 1, wherein detecting one or more characteristics of the patient comprises processing the one or more images using a machine-learning model for detecting dermatological conditions.

28. (canceled)

29. (canceled)

30. (canceled)

31. (canceled)

32. (canceled)

33. (canceled)

34. An interactive patient guidance system comprising:
a medical device;
a computing device comprising:
one or more cameras;
one or more indicators; and
one or more processors configured to run instructions to:
acquire one or more images of a patient via the one or more cameras;
detect one or more characteristics of the patient's body in the one or more images;
determine a target location for placing the medical device on the patient's body based on the detected one or more characteristics;
detect a location of the medical device;
generate guidance for moving the medical device from the detected location of the medical device to the determined target location; and
provide the guidance to the patient via the one or more indicators on the computing device.

35. The system of claim 34, wherein the computing device comprises a mirror.

36. (canceled)

37. (canceled)

38. (canceled)

39. (canceled)

40. The system of claim 34, wherein determining the target location comprises identifying one or more unsuitable areas of the patient's body based on the detected one or more characteristics.

41. The system of claim 40, wherein (i) the one or more unsuitable areas of the patient's body comprise areas with one or more of inflammation, infection, eczema, cancer, and psoriasis, and/or (ii) identifying the one or more unsuitable areas is based in part on how much time has elapsed since the patient last administered the medical device, and/or (iii) identifying the one or more unsuitable areas is based in part on orientation limitations of the medical device.

42. (canceled)

43. (canceled)

44. The system of claim 40, wherein determining the target location comprises generating a photogrammetric model of the patient and identifying the one or more unsuitable areas is based in part on the photogrammetric model.

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. A medical device comprising:
one or more indicators; and
one or more processors;
wherein the medical device is communicatively connected to an external processor and the one or more processors of the medical device are configured to:
receive one or more instructions from the external processor for providing guidance to a patient for placing the medical device on the patient's body; and
provide the guidance to the patient via the one or more indicators of the medical device.

68. (canceled)

69. (canceled)

70. (canceled)

71. (canceled)

72. (canceled)

73. (canceled)

74. (canceled)

75. (canceled)

76. (canceled)
